(19) United States
(12) Patent Application Publication
(10) Pub. No.: US 2025/0352567 A1
(43) Pub. Date: Nov. 20, 2025

(54) COMPOSITIONS COMPRISING A COMBINATION OF CAFFEINE AND OLEUROPEIN OR A METABOLITE THEREOF AND THEIR USE FOR IMPROVING MUSCLE FUNCTION

(71) Applicant: SOCIÉTÉ DES PRODUITS NESTLÉ S.A., Vevey (CH)

(72) Inventors: UMBERTO DE MARCHI, Corminboeuf (CH); JEROME FEIGE, Crissier (CH); ASTRID HORSTMAN, Bussigny (CH)

(21) Appl. No.: 18/866,329

(22) PCT Filed: May 16, 2023
(86) PCT No.: PCT/EP2023/063142

§ 371 Date: Nov. 15, 2024

(30) Foreign Application Priority Data
May 18, 2022 (EP) 22173984.0

(51) Int. Cl. A61K 31/7048; A61K 31/05; A61K 31/351; A61K 31/522; A61P 21/00

(52) U.S. Cl. CPC A61K 31/7048; A61K 31/05; A61K 31/351; A61K 31/522; A61P 21/00

(57) ABSTRACT

A combination of caffeine and at least one of oleuropein or metabolite thereof for use by oral administration to an individual in an amount effective to achieve at least one result that is (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue, (vi) increased mobility, and/or (vii) treatment of a muscle disorder linked to calcium depletion or deficiency. The individual can be at least one of an aging subject; an elderly subject; a subject with muscle fatigue or muscle weakness; a subject with impaired mobility; a frail subject; a pre-frail subject; a sarcopenic subject; a subject recovering from pre-frailty, frailty, sarcopenia or impaired mobility; a subject undergoing physical rehabilitation; a sportsman; or a pet.

### BACKGROUND

The present disclosure generally relates to compositions and methods that use a combination of caffeine and at least one of oleuropein or metabolite thereof. More specifically, the present disclosure relates to compositions and methods that increase bioenergetics and mitochondrial function through a combination of caffeine and at least one of oleuropein or metabolite thereof to boost mitochondrial calcium import, which in turn can increase muscle contraction and muscle performance to thereby improve, maintain or reduce loss of muscle functionality.

Sarcopenia is defined as the age-associated loss of muscle mass and functionality (including muscle strength and gait speed). Muscle functionality and physical ability decline with the loss of muscle mass. Impaired muscle functionality is highly predictive of the incidence of immobility, disability, and mortality in advanced age. With the rising elderly population, sarcopenia becomes increasingly prevalent such that 45% of the elderly U.S. population has moderate-to-severe symptoms. The U.S. health care direct and indirect costs attributable to sarcopenia reach nearly $19 billion. Therefore, prevention and/or treatment of sarcopenia would have a great impact on the health and quality of life of our society and consequently on the economy associated with health care. Unfortunately, the etiology and the physiopathological mechanism of sarcopenia are still poorly understood, making effective measures for prevention or treatment difficult.

### SUMMARY

Mitochondria are the primary source of aerobic energy production in mammalian cells and also maintain a large Ca2+ gradient across their inner membrane, providing a signaling potential for this molecule. Furthermore, mitochondrial Ca2+ plays a role in the mitochondria in the regulation of ATP generation and potentially contributes to the orchestration of cellular metabolic homeostasis. (Glancy, B. and R. S. Balaban (2012). “Role of mitochondrial Ca2+ in the regulation of cellular energetics.” Biochemistry 51(14): 2959-2973).

The present inventors noted that advancing age includes a gradual decrease in muscle function, capacity and reactivity. For example, a human aged 50 years loses about 10% of muscle area, and muscle strength declines by approximately 15% per decade in the ages of 60 and 70 years and by about 30% thereafter. Age-related decrease in muscle mass is responsible for almost all loss of strength and power in older adults, with an increase in fatigue. This decrease is due to inter-related factors: lifestyle, structural changes of the muscle, and metabolic changes.

The present inventors recognized this problem and addressed it by the surprising discovery that oleuropein and metabolites thereof are bioactives that activate mitochondrial calcium in combination with caffeine. Calcium is essential for skeletal muscle contraction, but there are very limited solutions to increase mitochondrial calcium uptake through natural bioactives in order to influence bioenergetics. Therefore, without being bound by theory, the present inventors believe that a combination of caffeine and at least one of oleuropein or metabolite thereof increases bioenergetics and mitochondrial function to boost mitochondrial calcium import, which in turn can increase muscle contraction and muscle performance to thereby improve, maintain or reduce loss of muscle functionality.

Accordingly, in a general embodiment, the present disclosure provides a method of achieving at least one result selected from the group consisting of (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue, (vi) increased mobility and (vii) treatment or prevention of a muscle disorder linked to calcium depletion or deficiency (e.g., reduction in incidence and/or severity). The method comprises orally administering to an individual an effective amount of a combination of caffeine and at least one of oleuropein or metabolite thereof.

In an embodiment, the present invention relates to a method of decreasing muscle fatigue in an individual who participates in exercise, the exercise comprising at least one of 1) resistance exercise, 2) anaerobic or repeated sprint-type exercise, or 3) endurance exercise, the method comprising orally administering to the individual an effective amount of a combination of caffeine and at least one of oleuropein or metabolite thereof.

In an embodiment, the individual is selected from the group consisting of an aging subject; an elderly subject; a subject with muscle fatigue or muscle weakness; a subject with impaired mobility; a frail subject; a pre-frail subject; a sarcopenic subject; a subject recovering from pre-frailty, frailty, sarcopenia or impaired mobility; a subject undergoing physical rehabilitation (e.g., from an injury to one or more of a muscle, a bone, a ligament, or the nervous system); a sportsman; and a pet.

In an embodiment, at least a portion of the muscle cells are part of a skeletal muscle selected from the group consisting of gastrocnemius, tibialis, soleus, extensor digitorum longus (EDL), biceps femoris, semitendinosus, semimembranosus, gluteus maximus, and combinations thereof.

In an embodiment, the combination of caffeine and at least one of oleuropein or metabolite thereof is orally administered daily for at least one week, preferably daily for at least one month.

In an embodiment, the metabolite of oleuropein is selected from the group consisting of oleuropein aglycone, hydroxytyrosol, homovanillyl alcohol, isohomovanillyl alcohol, glucuronidated forms thereof, sulfated forms thereof, derivatives thereof, and mixtures thereof.

In an embodiment, the combination of caffeine and at least one of oleuropein or metabolite thereof is administered in a composition selected from the group consisting of food compositions, dietary supplements, nutritional compositions, beverages, nutraceuticals, powdered nutritional products to be reconstituted in water or milk before consumption, food additives, medicaments, drinks, petfood and combinations thereof.

In an embodiment, the caffeine and the at least one of oleuropein or metabolite thereof are administered together in the same composition.

In an embodiment, the caffeine is administered separately in a different composition from the at least one of oleuropein or metabolite thereof.

In an embodiment, the caffeine and the at least one of oleuropein or metabolite thereof are administered together in a food product further comprising a component selected from the group consisting of protein, carbohydrate, fat and mixtures thereof.

In another embodiment, the present disclosure provides a method of treating in an individual in need thereof or preventing in an individual at risk thereof (e.g., reducing incidence and/or severity) at least one condition selected from the group consisting of (i) impairment in at least one of muscle functionality, muscle performance, or muscle strength, (ii) muscle fatigue or muscle weakness, (iii) pre-frailty, frailty, sarcopenia or impaired mobility, and (iv) a muscle disorder linked to calcium depletion or deficiency. The method comprises orally administering to the individual in need thereof or at risk thereof an effective amount of a combination of caffeine and at least one of oleuropein or metabolite thereof.

In another embodiment, the present disclosure provides a unit dosage form comprising a combination of caffeine and at least one of oleuropein or metabolite thereof, the unit dosage form comprises an amount of the combination effective for at least one result selected from the group consisting of (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue, (vi) increased mobility and (vii) treatment or prevention of a muscle disorder linked to calcium depletion or deficiency (e.g., reduction in incidence and/or severity).

In an embodiment, the unit dosage form consists essentially of the combination of caffeine and at least one of oleuropein or metabolite thereof.

In an embodiment, the unit dosage form consists of an excipient and the combination of caffeine and at least one of oleuropein or metabolite thereof.

In another embodiment, the present disclosure provides a method of making a composition for achieving at least one result selected from the group consisting of (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue or muscle weakness, (vi) increased mobility and (vii) treatment or prevention of a muscle disorder linked to calcium depletion or deficiency (e.g., reduction in incidence and/or severity). The method comprises adding an effective amount of a combination of caffeine and at least one of oleuropein or metabolite thereof to at least one ingredient selected from the group consisting of protein, carbohydrate, and fat.

In an embodiment, the method further comprises adding to the at least one ingredient a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof.

Additional features and advantages are described herein and will be apparent from the following Figures and Detailed Description.

### BRIEF DESCRIPTION OF DRAWINGS

FIG. 1 is a graph showing that effect of oleuropein aglycone (10 μM, gray), caffeine (5 mM, black) and the combination of 10 μM oleuropein aglycone+5 mM caffeine on mitochondrial activation, measured as integrated mitochondrial calcium rise, in myotubes differentiated from C2C12 cells. Results are expressed as mean+/−SEM from n=29 experiments. * indicates statistically significant difference of at P<0.05 (one-way ANOVA test).

FIG. 2 is a graph showing that oleuropein (10 μM) synergizes with caffeine (5 mM) to promote mitochondrial calcium rise, in myotubes from C2C12 cells. The graph, obtained from the data represented in FIG. 1, compares the theoretical additive effect of caffeine supplementation plus oleuropein aglycone supplementation with the measured effect of the combination of caffeine plus oleuropein aglycone simultaneously supplemented, on mitochondrial calcium rise. The theoretical effect is compared with the measured effect to extrapolate the synergism. Data are the average of 29 experiments. Results are expressed as mean+/−SEM. * indicates statistically significant difference of the measured vs. theoretical difference in mitochondrial calcium at P<0.05 (Student's t-test).

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a metabolite” or “the metabolite” includes one metabolite but also two or more metabolites.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

As used herein, a “composition consisting essentially of a combination of calcium and at least one of oleuropein or metabolite thereof” does not include any additional compound that affects mitochondrial calcium import other than the combination of calcium and at least one of oleuropein or metabolite thereof. In a particular non-limiting embodiment, the composition consists of an excipient and the combination of calcium and at least one of oleuropein or metabolite thereof.

The term “and/or” used in the context of “X and/or Y” should be interpreted as “X,” or “Y,” or “X and Y.” Similarly, “at least one of X or Y” should be interpreted as “X,” or “Y,” or “both X and Y.” For example, “at least one of oleuropein or metabolite thereof” means “oleuropein,” or “a metabolite of oleuropein,” or “both oleuropein and a metabolite thereof.”

Where used herein, the terms “example” and “such as,” particularly when followed by a listing of terms, are merely exemplary and illustrative and should not be deemed to be exclusive or comprehensive. As used herein, “associated with” and “linked with” mean occurring concurrently, preferably means caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development or progression of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

Both human and veterinary treatments are within the scope of the present disclosure. Preferably the combination of caffeine and at least one of oleuropein or metabolite thereof is administered in a serving or unit dosage form that provides a therapeutically effective or prophylactically effective amount of the combination.

The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder.

As used herein, an “effective amount” is an amount that treats or prevents a deficiency, treats or prevents a disease or medical condition in an individual, or, more generally, reduces symptoms, manages progression of the disease, or provides a nutritional, physiological, or medical benefit to the individual.

The relative terms “improved,” “increased,” “enhanced” and the like refer to the effects of the composition disclosed herein, namely a composition comprising an effective amount of a combination of caffeine and at least one of oleuropein or metabolite thereof, relative to administration over the same time period of a composition lacking one of the caffeine or the oleuropein/oleuropein metabolite but otherwise identical.

As used herein, “administering” includes another individual providing a referenced composition to an individual so that the individual can consume the composition and also includes merely the act of the individual themselves consuming a referenced composition.

“Animal” includes, but is not limited to, mammals, which includes but is not limited to rodents; aquatic mammals; domestic animals such as dogs, cats and other pets; farm animals such as sheep, pigs, cows and horses; and humans. Where “animal,” “mammal” or a plural thereof is used, these terms also apply to any animal that is capable of the effect exhibited or intended to be exhibited by the context of the passage, e.g., an animal benefitting from improved mitochondrial calcium import. While the term “individual” or “subject” is often used herein to refer to a human, the present disclosure is not so limited. Accordingly, the term “individual” or “subject” refers to any animal, mammal or human that can benefit from the methods and compositions disclosed herein.

The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal. The term “companion animal” means a dog or a cat.

The term “elderly” in the context of a human means an age from birth of at least 60 years, preferably above 63 years, more preferably above 65 years, and most preferably above 70 years. In the context of non-human animals, “elderly” means a non-human subject that has reached 60% of its likely lifespan, in some embodiments at least 70%, at least 80% or at least 90% of its likely lifespan. A determination of lifespan may be based on actuarial tables, calculations, or estimates, and may consider past, present, and future influences or factors that are known to positively or negatively affect lifespan. Consideration of species, gender, size, genetic factors, environmental factors and stressors, present and past health status, past and present nutritional status, and stressors may be taken into consideration when determining lifespan.

The term “older adult” in the context of a human means an age from birth of at least 45 years, preferably above 50 years, more preferably above 55 years, and includes elderly individuals.

“Mobility” is the ability to move independently and safely from one place to another.

“Sarcopenia” is defined as the age-associated loss of muscle mass and functionality (including muscle strength and gait speed).

As used herein, “frailty” is defined as a clinically recognizable state of increased vulnerability resulting from aging-associated decline in reserve and function across multiple physiologic systems such that the ability to cope with everyday or acute stressors is compromised. In the absence of an established quantitative standard, frailty has been operationally defined by Fried et al. as meeting three out of five phenotypic criteria indicating compromised energetics: (1) weakness (grip strength in the lowest 20% of population at baseline, adjusted for gender and body mass index), (2) poor endurance and energy (self-reported exhaustion associated with \dot{V}O2 max), (3) slowness (lowest 20% of population at baseline, based on time to walk 15 feet, adjusting for gender and standing height), (4) low physical activity (weighted score of kilocalories expended per week at baseline, lowest quintile of physical activity identified for each gender; e.g., less than 383 kcal/week for males and less than 270 kcal/week for females), and/or unintentional weight loss (10 lbs. in past year). Fried L P, Tangen C M, Walston J, et al., “Frailty in older adults: evidence for a phenotype.” J. Gerontol. A. Biol. Sci. Med. Sci. 56(3):M146-M156 (2001). A pre-frail stage, in which one or two of these criteria are present, identifies a high risk of progressing to frailty.

“Muscle fatigue” means a reduced contractile force in one or more muscles due to a shortage of substrates within the muscle fiber and/or an accumulation of metabolites within the muscle fiber which interfere either with the release of calcium or with the ability of calcium to stimulate muscle contraction.

“Muscle weakness” is a condition where the force exerted by the muscles is less than would be expected. The U.S. Medical Research Council's grading system for muscle strength is widely used to identify muscle weakness and the severity thereof. Specifically, the examiner assesses the patient's ability to move the muscle against resistance provided by the examiner who, through experience, has developed a sense of the expected range of normal. This will vary from patient-to-patient depending upon the underlying size and conditioning of the subject; the fully trained athlete can be expected to perform differently from a small, sedentary, or deconditioned individual. The expected strength should also be adjusted for degree of atrophy in patients with wasting illnesses.

The patient's effort is graded on a scale of 0 to 5. As used herein, “muscle weakness” refers to any of grades 0-4.
Grade 5: Muscle contracts normally against full resistance.
Grade 4: Muscle strength is reduced, but muscle contraction can still move joint against resistance.
Grade 3: Muscle strength is further reduced, such that the joint can be moved only against gravity with the examiner's resistance completely removed. As an example, the elbow can be moved from full extension to full flexion starting with the arm hanging down at the side.
Grade 2: Muscle can move only if the resistance of gravity is removed. As an example, the elbow can be fully flexed only if the arm is maintained in a horizontal plane.
Grade 1: Only a trace or flicker of movement is seen or felt in the muscle, or fasciculations are observed in the muscle.
Grade 0: No movement is observed.

As used herein, a “sportsman” is an individual who participates in at least one of 1) resistance exercise, 2) anaerobic or repeated sprint-type exercise, or 3) endurance exercise.

Resistance exercise is when a subject undertakes explosive movements of weight, with long periods of rest, and is primarily driven by the phosphocreatine and glycolytic energy systems. Resistance exercise can produce energy quickly, but the subject fatigues quickly. The primary adaptations include increases in muscle mass (hypertrophy) by increased muscle cross-section area through repeated weight lifting training. Hakkinen K. 1989. Neuromuscular and hormonal adaptations during strength and power training. J. Sports Med. Phys. Fitness. 29:9-26; and Hakkinen K. et. al. 1987. Relationships between training volume, physical performance capacity, and serum hormone concentrations during prolonged training in elite weight lifters. Int. J. Sports Med. 8 Suppl 1:61-65.

Repeated sprint-type training is anaerobic, involves high-intensity exercise with limited recovery periods, and involves nearly purely carbohydrate metabolism with a large breakdown in muscle glycogen (glycolytic energy production). During these situations of anaerobic energy production, such as high intensity speed training or sports involving repeated sprints, the increased load on the muscles is accomplished by an increased firing of Type IIa fibers. Finally, at very high workloads, type IIb glycolytic muscle fibers become activated to maintain the high demand of energy provision via anaerobic energy provision. However, during these situations, the high rate of anaerobic energy production exceeds the rate at which it can be oxidized aerobically within the mitochondria, and this leads to the extreme levels of lactate production found in these types of training situations. Spriet L L, Howlett R A, and Heigenhauser G J. 2000. An enzymatic approach to lactate production in human skeletal muscle during exercise. Med. Sci. Sports Exerc. 32: 756-763.

Endurance training is characterized by individuals performing low-intensity training over prolonged periods (e.g., >15 minutes). The energy system represented for endurance training includes the aerobic system, which primarily uses aerobic metabolism of fats and carbohydrates to produce the required energy within the mitochondria when ample oxygen is present. The primary adaptations include increased muscle glycogen stores and glycogen sparing at sub-maximal workloads via increased fat oxidation, enhanced lactate kinetics and morphological alterations, including greater type I fiber per muscle area, and increased capillary and mitochondrial density. Holloszy J O, and Coyle E F. 1984. Adaptations of skeletal muscle to endurance exercise and their metabolic consequences. J. Appl. Physiol. 56: 831-838; and Holloszy J O, Rennie M J, Hickson R C, Conlee R K, and Hagberg J M. 1977. Physiological consequences of the biochemical adaptations to endurance exercise. Ann. N.Y. Acad. Sci. 301: 440-450.

The terms “serving” or “unit dosage form,” as used herein, are interchangeable and refer to physically discrete units suitable as unitary dosages for human and animal subjects, each unit containing a predetermined quantity of the composition comprising a combination of calcium and at least one of oleuropein or metabolite thereof, as disclosed herein, in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of liquid housed within a container such as a bottle.

An “oral nutrition supplement” or “ONS” is a composition comprising at least one macronutrient and/or at least one micronutrient, for example in a form of sterile liquids, semi-solids or powders, and intended to supplement other nutritional intake such as that from food. Non-limiting examples of commercially available ONS products include MERITENE®, BOOST®, NUTREN® and SUSTAGEN®. In some embodiments, an ONS can be a beverage in liquid form that can be consumed without further addition of liquid, for example an amount of the liquid that is one serving of the composition.

As used herein, “incomplete nutrition” refers to preferably nutritional products that do not contain sufficient levels of macronutrients (protein, fats and carbohydrates) or micronutrients to be sufficient to be a sole source of nutrition for the animal to which the nutritional product is being administered. The term “complete nutrition” refers to a product which is capable of being the sole source of nutrition for the subject. An individual can receive 100% of their nutritional requirements from a complete nutrition composition.

A “kit” means that the components of the kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof.

### EMBODIMENTS

An aspect of the present disclosure is a method of achieving at least one result selected from the group consisting of (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue or muscle weakness, (vi) increased mobility and (vii) treatment or prevention of a muscle disorder linked to calcium depletion or deficiency (e.g., reduction in incidence and/or severity). The method comprises orally administering to an individual an effective amount of a combination of caffeine and at least one of oleuropein or metabolite thereof.

Another aspect of the present disclosure is a method of treating in an individual in need thereof or preventing in an individual at risk thereof (e.g., reducing incidence and/or severity) at least one condition selected from the group consisting of (i) impairment in at least one of muscle functionality, muscle performance, or muscle strength, (ii) muscle fatigue or muscle weakness, (iii) pre-frailty, frailty, sarcopenia or impaired mobility, and (iv) a muscle disorder linked to calcium depletion or deficiency. The method comprises orally administering to the individual in need thereof or at risk thereof an effective amount of a combination of caffeine and at least one of oleuropein or metabolite thereof.

This also results in an improved vitality and/or energy in the individual.

In another embodiment, decreasing muscle fatigue is in an individual who participates in exercise, the exercise comprising at least one of 1) resistance exercise, 2) anaerobic or repeated sprint-type exercise, or 3) endurance exercise, the method comprising orally administering to the individual an effective amount of a combination of caffeine and at least one of oleuropein or metabolite thereof. The combination of caffeine and at least one of oleuropein or metabolite thereof is administered to the individual before the exercise, and/or during the exercise, and/or after the exercise, preferably less than two hours before the exercise, and/or during the exercise, and/or less than two hours after the exercise. In a more preferred embodiment, the combination of caffeine and at least one of oleuropein or metabolite thereof is administered to the individual less than one hour before the exercise.

The effective amount of the combination of caffeine and at least one of oleuropein or metabolite thereof varies with the particular composition, the age and condition of the recipient, and the particular disorder or disease being treated. Nevertheless, in a general embodiment, 0.001 mg to 1.0 g of the at least one of oleuropein or metabolite thereof can be administered to the individual per day, preferably from 0.01 mg to 0.9 g of the at least one of oleuropein or metabolite thereof per day, more preferably from 0.1 mg to 750 mg of the at least one of oleuropein or metabolite thereof per day, more preferably from 0.5 mg to 500 mg of the at least one of oleuropein or metabolite thereof per day, and most preferably from 1.0 mg to 200 mg of the at least one of oleuropein or metabolite thereof per day.

In an embodiment, at least a portion of the oleuropein is obtained by extraction, e.g., by extraction from a plant such as a plant belonging to the Oleaceae family, preferably one or more of the stems, the leaves, the fruits or the stones of a plant belonging to the Oleaceae family such as Olea europaea (olive tree), a plant of genus Ligustrum, a plant of genus Syringa, a plant of genus Fraximus, a plant of genus Jasminum and a plant of genus Osmanthus. Additionally or alternatively, at least a portion of the oleuropein can be obtained by chemical synthesis.

Non-limiting examples of suitable metabolites of oleuropein include oleuropein aglycone, hydroxytyrosol, homovanillyl alcohol, isohomovanillyl alcohol, and mixtures thereof.

In some embodiments, the at least one of oleuropein or metabolite thereof is the only polyphenol in the composition and/or the only polyphenol administered to the individual.

The effective amount of caffeine also varies with the particular composition, the age and condition of the recipient, and the particular disorder or disease being treated. Nevertheless, in a general embodiment, up to 400 mg of caffeine can be administered to the individual per day, preferably from about 20 mg to about 380 mg, more preferable from about 40 to about 300 mg per day. Alternatively, caffeine may also be present in the composition in an amount of about 0.05-0.3 g/serving, about 0.1-0.2 g/serving, about 0.125-0.175 g/serving, or about 0.14 g/serving.

In some embodiments, the combination of caffeine and at least one of oleuropein or metabolite thereof is administered to an individual selected from the group consisting of an aging subject; an elderly subject; a subject with muscle fatigue or muscle weakness; a subject with impaired mobility; a frail subject; a pre-frail subject; a sarcopenic subject; a subject recovering from pre-frailty, frailty, sarcopenia or impaired mobility; a subject undergoing physical rehabilitation (e.g., from an injury to one or more of a muscle, a bone, a ligament, or the nervous system); a sportsman; and a pet. In some embodiments, the individual is healthy. In some embodiments, the individual has sarcopenia, frailty, muscle fatigue or muscle weakness, or impairment in one or more of muscle functionality, muscle performance, or muscle strength, but optionally is otherwise healthy.

For example, the combination of caffeine and at least one of oleuropein or metabolite thereof can be administered to a sportsman before, during and/or after exercise, for example less than two hours before the exercise or less than one hour before the exercise and less than two hours after the exercise or less than one hour after the exercise.

In an embodiment, at least a portion of the muscle cells are part of a skeletal muscle selected from the group consisting of gastrocnemius, tibialis, soleus, extensor digitorum longus (EDL), biceps femoris, semitendinosus, semimembranosus, gluteus maximus, and combinations thereof.

The combination of caffeine and at least one of oleuropein or metabolite thereof can be administered in any composition that is suitable for human and/or animal consumption. In a preferred embodiment, the combination of caffeine and at least one of oleuropein or metabolite thereof is administered to the individual orally or enterally (e.g. tube feeding). For example, the combination of caffeine and at least one of oleuropein or metabolite thereof can be administered to the individual in a beverage, a food product, a capsule, a tablet, a powder or a suspension.

Non-limiting examples of suitable compositions for the include food compositions, dietary supplements, dietary supplements (e.g., liquid ONS), complete nutritional compositions, beverages, pharmaceuticals, nutraceuticals, powdered nutritional products to be reconstituted in water or milk before consumption, food additives, medicaments, drinks, petfood and combinations thereof.

Food products according to the present invention may include dairy products, such as fermented milk products, e.g., yoghurts, buttermilk, etc; ice creams; concentrated milk; milk; dairy creams; flavoured milk drinks; whey based drinks; toppings; coffee creamers; chocolate; cheese based products; soups; sauces; purees; dressings; puddings; custards; baby foods; nutritional formulas, such as those for complete nutrition, for example for infants, children, teenagers, adults, the elderly or the critically ill; cereals and cereal bars, for example.

Drinks may include for example milk- or yoghurt based drinks, fermented milk, protein drinks, coffee, tea, energy drinks, soy drinks, fruit and/or vegetable drinks, fruit and/or vegetable juices.

The combination of caffeine and at least one of oleuropein or metabolite thereof can be administered in a food product further comprising a component selected from the group consisting of protein, carbohydrate, fat and mixtures thereof.

In some instances where oral or enteral administration is not possible or not advised, the composition may be administered parenterally.

Preferably, the muscle functionality that can be improved by the methods disclosed herein comprises a characteristic selected from the group consisting of muscle strength, gait speed, and combinations thereof. Muscle function is typically defined as strength per unit of appendicular skeletal muscle mass or per muscle volume.

Non-limiting examples of a muscle disorder linked to calcium depletion or deficiency that can be treated by the methods disclosed herein include muscular dystrophies, congenital core myopathies and mitochondrial myopathies. Particular non-limiting examples include Barth syndrome; chronic progressive external ophthalmoplegia (cPEO); Kearns-Sayre syndrome (KSS); Leigh syndrome; mitochondrial DNA depletion syndromes (MDDS); mitochondrial encephalomyopathy, lactic acidosis, and stroke-like episodes (MELAS); mitochondrial neurogastrointestinal encephalomyopathy (MNGIE); myoclonus epilepsy with ragged red fibers (MERRF); neuropathy, ataxia, and retinitis pigmentosa (NARP); and Pearson syndrome.

The individual can be at risk of a disorder or condition (e.g., sarcopenia, frailty, muscle fatigue or muscle weakness, or impairment in one or more of muscle functionality, muscle performance, or muscle strength), in which case the effective amount of the composition is a prophylactically effective dose; or the individual can have a disorder or condition, in which case the effective amount of the composition is a therapeutically effective dose. In some embodiments, the methods comprise identifying the individual as having the condition or being at risk of the condition before the administration.

In another embodiment, the present disclosure provides a method of treating or preventing impaired mobility in an older adult. The method comprises orally administering to the older adult an effective amount of a combination of caffeine and at least one of oleuropein or metabolite thereof. The older adult can be an elderly individual. In some embodiments, the older adult has a condition selected from the group consisting of frailty, pre-frailty, sarcopenia, recovering from sarcopenia, osteoporosis, osteoarthritis, malnutrition, at risk of malnutrition, undergoing rehabilitation, scheduled to undergo rehabilitation within the next year, and combinations thereof.

The composition may be administered to the older adult in an amount sufficient to prevent, at least partially reduce the risk of developing frailty or sarcopenia, and/or at least partially reduce the severity of pre-frailty, frailty, sarcopenia or impaired mobility in instances where the condition has yet not been developed in the individual. Such an amount is defined to be “a prophylactically effective dose.” Again, the precise amounts depend on a number of factors relating to the individual, such as their weight, health and how much muscle functionality (e.g., muscle strength, gait speed, etc.) is being lost.

In an embodiment, the combination of caffeine and at least one of oleuropein or metabolite thereof is administered to the individual for a time period of at least one month; preferably at least two months, more preferably at least three, four, five or six months; most preferably for at least one year. During the time period, the combination of caffeine and at least one of oleuropein or metabolite thereof can be administered to the individual at least one day per week; preferably at least two days per week, more preferably at least three, four, five or six days per week; most preferably seven days per week. The combination of caffeine and at least one of oleuropein or metabolite thereof can be administered in a single dose per day or in multiple separate doses per day.

The above examples of administration do not require continuous daily administration with no interruptions. Instead, there may be some short breaks in the administration, such as a break of two to four days during the period of administration. The ideal duration of the administration of the composition can be determined by those of skill in the art.

In an embodiment, the caffeine and the at least one of oleuropein or metabolite thereof can be administered in the same composition, for example a unit dosage form containing both the caffeine and the at least one of oleuropein or metabolite thereof.

In an alternative embodiment, the caffeine and the at least one of oleuropein or metabolite thereof can be administered sequentially in separate compositions. The term “sequentially” means that the caffeine and the at least one of oleuropein or metabolite thereof are administered in a successive manner such that the at least one of oleuropein or metabolite thereof is administered at a first time without the caffeine, and the caffeine is administered at a second time (before or subsequent to the first time) without the at least one of oleuropein or metabolite thereof. The time between sequential administrations may be, for example, one or several seconds, minutes or hours in the same day; one or several days or weeks in the same month; or one or several months in the same year.

Another aspect of the present disclosure is a method of making a composition for achieving an effect selected from the group consisting of (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue, (vi) increased mobility and (vii) treatment of a muscle disorder linked to calcium depletion or deficiency.

The method comprises adding a combination of caffeine and at least one of oleuropein or metabolite thereof to an ingredient selected from the group consisting of a protein, a carbohydrate, a lipid, and combinations thereof. The composition (e.g., food product) can be made prior to administration (e.g., the composition is made, packaged, and then purchased by a consumer who administers the composition to themselves or to another individual) or can be made substantially simultaneous to administration (the composition is made less than 30 minutes before administration, preferably less than 15 minutes before administration, more preferably less than 10 minutes before administration, most preferably less than 5 minutes before administration, by an individual who administers the composition to themselves or to another individual).

The composition can comprise an effective amount of the combination of caffeine and at least one of oleuropein or metabolite thereof. For example, a single serving or dose of the composition can comprise the effective amount of the combination, and a package can contain one or more of the servings or doses.

The composition can comprise a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof.

In addition to the combination of caffeine and at least one of oleuropein or metabolite thereof, the composition can further comprise a protein source from animal or plant origin, for example milk proteins, soy proteins, and/or pea proteins. In a preferred embodiment, the protein source is selected from the group consisting of whey protein; casein protein; pea protein; soy protein; wheat protein; corn protein; rice protein; proteins from legumes, cereals and grains; and combinations thereof. Additionally or alternatively, the protein source may comprise a protein from nuts and/or seeds.

The protein source preferably comprises whey protein. The whey protein may be unhydrolyzed or hydrolyzed whey protein. The whey protein may be any whey protein, for example the whey protein can be selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof. In a preferred embodiment, the whey protein comprises whey protein isolate and/or modified sweet whey.

As noted above, the protein source can be from animal or plant origin, for example milk proteins, soy proteins, and/or pea proteins. In an embodiment, the protein source comprises casein. Casein may be obtained from any mammal but is preferably obtained from cow milk and preferably as micellar casein.

The composition can comprise one or more branched chain amino acids. For example, the composition can comprise leucine, isoleucine and/or valine. The protein source in the composition may comprise leucine in free form and/or leucine bound as peptides and/or proteins such as dairy, animal or vegetable proteins. In an embodiment, the composition comprises the leucine in an amount up to 10 wt % of the dry matter of the composition. Leucine can be present as D- or L-leucine and preferably the L-form. If the composition comprises leucine, the composition can be administered in a daily dose that provides 0.01 to 0.04 g of the leucine per kg body weight, preferably 0.02 to 0.035 g of the leucine per kg body weight. Such doses are particularly applicable to complete nutrition compositions, but one of ordinary skill will readily recognize how to adapt these doses for an oral nutritional supplement (ONS).

One or more other minerals can be used in the composition. Non-limiting examples of suitable minerals include calcium, boron, chromium, copper, iodine, iron, magnesium, manganese, molybdenum, nickel, phosphorus, potassium, selenium, silicon, tin, vanadium, zinc, and combinations thereof.

One or more other vitamins additional to any can be used in the composition. Non-limiting examples of suitable vitamins include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), Vitamin C, Vitamin D, Vitamin E, Vitamin K, folic acid and biotin), and combinations thereof. “Vitamin” includes such compounds obtained naturally from plant and animal foods or synthetically made, pro-vitamins, derivatives thereof, and analogs thereof.

The composition may also contain a carbohydrate and/or a source of fat. Non-limiting examples of suitable fats include canola oil, corn oil and high-oleic acid sunflower oil. Non-limiting examples of suitable carbohydrates include sucrose, lactose, glucose, fructose, corn syrup solids, maltodextrins, and mixtures thereof. Additionally or alternatively, a dietary fiber may be added. Dietary fiber passes through the small intestine undigested by enzymes and functions as a natural bulking agent and laxative. Dietary fiber may be soluble or insoluble and generally a blend of the two types is preferred. Non-limiting examples of suitable dietary fibers include soy, pea, oat, pectin, guar gum, partially hydrolyzed guar gum, gum Arabic, fructo-oligosaccharides, acidic oligosaccharides, galacto-oligosaccharides, sialyl-lactose and oligosaccharides derived from animal milks. A preferred fiber blend is a mixture of inulin with shorter chain fructo-oligosaccharides. In an embodiment, the fiber content is between 2 and 40 g/L of the composition, for example between 4 and 10 g/L.

One or more food grade emulsifiers may be incorporated into the composition, such as diacetyl tartaric acid esters of mono- and di-glycerides, lecithin, and/or mono- and di-glycerides. Suitable salts and stabilizers may be included.

### Examples

The following non-limiting examples present experimental data supporting the compositions and methods disclosed herein.

### Example 1

To test the synergism of the combined effect of oleuropein+caffeine on mitochondrial calcium rise, the inventors measured mitochondrial calcium rise in myotubes differentiated form C2C12 cells. C2C12 cells were purchased from ATCC. C2C12 cells were seeded in 96-well plates at a density of 8000 cells per well in DMEM high glucose (Gibco)+10% fetal calf serum. Myotubes were differentiated from C2C12 cells by growing the cells in DMEM containing 2% horse serum, for 4 days. Mitochondrial calcium measurements were carried out using myotubes infected with the adenovirus (from Sirion biotech) expressing the mitochondrially targeted calcium sensor mitochondrial mutated aequorin (Montero et al., 2004). For aequorin reconstitution, 24 hours after infection, myotubes were incubated for 2 h at room temperature (22° C.) in standard medium (145 mM NaCl, 5 mM KCl, 1 mM MgCl₂, 1 mM CaCl₂, 10 mM glucose and 10 mM Hepes, pH 7.4) with 1 μM wild-type coelenterazine. For treatment, compounds were directly added to the cell culture or myotubes cultures 2 hours before measurements. Luminescence was measured at the FLIPR Tetra Aequorin (Molecular Devices). Calibration of the luminescence data into calcium concentration was carried out using an algorithm as described previously (Alvarez & Montero, 2002). Custom module analysis based on Excel (Microsoft) and GraphPad Prism 7.02 (GraphPad) software was used for quantification

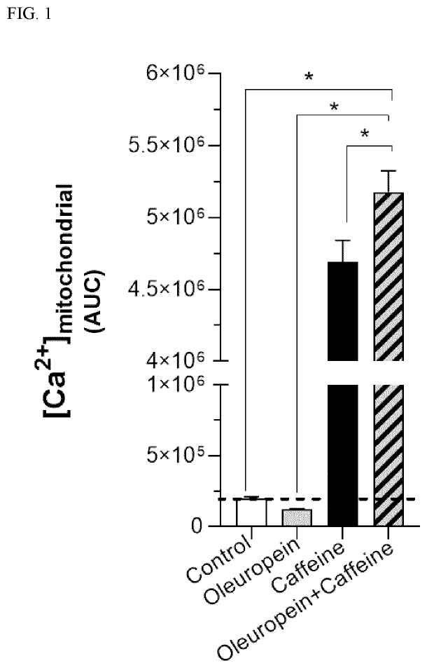

As shown in FIG. 1, the simultaneous supplementation of oleuropein aglycone and caffeine causes a mitochondrial activation (measured as mitochondrial calcium elevation) bigger than the effect of each compound alone,

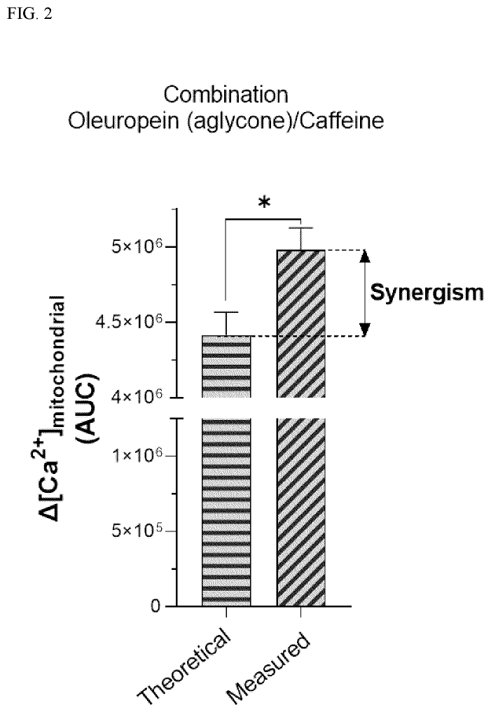

As shown in the main FIG. 2, to calculate the synergistic effect of oleuropein aglycone+caffeine, the theoretical effect of the sum of the 2 distinct compounds (oleuropein aglycone and caffeine) was compared with the measured effect of the combination. As shown in FIG. 2 oleuropein synergizes with caffeine to promote mitochondrial calcium rise during stimulation.

### REFERENCES

Glancy, B. and R. S. Balaban (2012). “Role of mitochondrial Ca2+ in the regulation of cellular energetics.” Biochemistry 51(14), 2959-2973.
Alvarez, J., & Montero, M. (2002). Measuring [Ca2+] in the endoplasmic reticulum with aequorin. Cell Calcium, 32(5-6), 251-260.
Montero, M., Lobaton, C. D., Hernandez-Sanmiguel, E., Santodomingo, J., Vay, L., Moreno, A., & Alvarez, J. (2004). Direct activation of the mitochondrial calcium uniporter by natural plant flavonoids. Biochem J, 384(Pt 1), 19-24. doi:10.1042/BJ20040990.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of achieving at least one result selected from the group consisting of (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue, (vi) increased mobility and (vii) treatment of a muscle disorder linked to calcium depletion or deficiency, the method comprising orally administering to an individual an effective amount of a combination of caffeine and at least one of oleuropein or metabolite thereof.

2. The method of claim 1, wherein the individual is selected from the group consisting of an aging subject; an elderly subject; a subject with muscle fatigue or muscle weakness; a subject with impaired mobility; a frail subject; a pre-frail subject; a sarcopenic subject; a subject recovering from pre-frailty, frailty, sarcopenia or impaired mobility; a subject undergoing physical rehabilitation; a sportsman; and a pet.

3. The method of claim 1, wherein decreasing muscle fatigue is in an individual who participates in exercise, the exercise comprising at least one of 1) resistance exercise, 2) anaerobic or repeated sprint-type exercise, or 3) endurance exercise, the method comprising orally administering to the individual an effective amount of a combination of caffeine and at least one of oleuropein or metabolite thereof.

4. The method of claim 3, wherein the combination of caffeine and at least one of oleuropein or metabolite thereof is administered to the individual before the exercise, and/or during the exercise, and/or after the exercise.

5. The method of claim 1, wherein at least a portion of the muscle cells are part of a skeletal muscle selected from the group consisting of gastrocnemius, tibialis, soleus, extensor digitorum longus (EDL), biceps femoris, semitendinosus, semimembranosus, gluteus maximus, and combinations thereof.

6. The method of claim 1, wherein the combination of caffeine and at least one of oleuropein or metabolite thereof is administered daily for at least one week.

7. The method of claim 1, wherein the metabolite of oleuropein is selected from the group consisting of oleuropein aglycone, hydroxytyrosol, homovanillyl alcohol, isohomovanillyl alcohol, glucuronidated forms thereof, sulfated forms thereof, derivatives thereof, and mixtures thereof.

8. The method of claim 1, wherein the combination of caffeine and at least one of oleuropein or metabolite thereof is administered in a composition selected from the group consisting of food compositions, dietary supplements, nutritional compositions, beverages, nutraceuticals, powdered nutritional products to be reconstituted in water or milk before consumption, food additives, medicaments, drinks, petfood, and combinations thereof.

9. The method of claim 1, wherein the caffeine and the at least one of oleuropein or metabolite thereof are administered together in the same composition.

10. The method of claim 1, wherein the caffeine is administered separately in a different composition from the at least one of oleuropein or metabolite thereof.

11. The method of claim 1, wherein the caffeine and the at least one of oleuropein or metabolite thereof are administered together in a food product further comprising a component selected from the group consisting of protein, carbohydrate, fat and mixtures thereof.

12. A method of treating in an individual in need thereof or preventing in an individual at risk thereof at least one condition selected from the group consisting of (i) impairment in at least one of muscle functionality, muscle performance, or muscle strength, (ii) muscle fatigue or muscle weakness, (iii) pre-frailty, frailty, sarcopenia or impaired mobility, and (iv) a muscle disorder linked to calcium depletion or deficiency, the method comprising orally administering to the individual in need thereof or at risk thereof an effective amount of a combination of caffeine and at least one of oleuropein or metabolite thereof.

13-15. (canceled)

16. A method of making a composition for achieving at least one result selected from the group consisting of (i) improved mitochondrial calcium uptake in muscle cells, (ii) improved utilization of calcium in muscle cells, (iii) increased mitochondrial energy in muscle cells, (iv) improvement in at least one of muscle functionality, muscle performance, or muscle strength, (v) decreased muscle fatigue or muscle weakness, (vi) increased mobility and (vii) treatment of a muscle disorder linked to calcium depletion or deficiency, the method comprising adding an effective amount of a combination of caffeine and at least one of oleuropein or metabolite thereof to at least one ingredient selected from the group consisting of protein, carbohydrate, and fat.

17. The method of claim 16 further comprising adding to the at least one ingredient a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof.
